(19) United States
(12) Patent Application Publication
(10) Pub. No.: US 2021/0401353 A1
(43) Pub. Date: Dec. 30, 2021

(54) Absence of Suspicion Test

(71) Applicant: University of Cincinnati, Cincinnati, OH (US)

(72) Inventors: Joseph F Clark, Cincinnati, OH (US); Jon Vincent, Loveland, OH (US)

(73) Assignee: University of Cincinnati, Cincinnati, OH (US)

(21) Appl. No.: 17/357,246

(22) Filed: Jun. 24, 2021

Related U.S. Application Data
(60) Provisional application No. 63/043,274, filed on Jun. 24, 2020.

(51) Int. Cl. A61B 5/00; A61B 3/09; A61B 3/06; A61B 3/113

(52) U.S. Cl. CPC A61B 5/4064; A61B 3/09; A61B 2503/10; A61B 3/113; A61B 3/063

(57) ABSTRACT

A method of determining that an absence of suspicion of neurological injury is indicated in a subject is disclosed. The method involves prompting the subject to engage in three or more of the following neurological assessments: a) Reading Saccades, b) Near/far accommodation using Hart charts of different font sizes; c) Brock string, d) Maddox rod, e) closed-eye turns, f) Near Point of Convergence, and g) PERRLA with consensual reflex. The data from the neurological assessments is used to determine that an absence of suspicion of neurological injury is indicated in the subject.

### CROSS REFERENCE TO RELATED APPLICATIONS

The application claims priority to U.S. Provisional Application Ser. No. 63/043,274, filed Jun. 24, 2020, which application is hereby incorporated by reference in its entirety.

### TECHNICAL FIELD

The present invention relates to assessment tools to aid with concussion detection.

### BACKGROUND OF THE INVENTION

Concussions are reported by the American Medical Society for Sports Medicine as a serious health concern among healthcare professionals, athletic organizations, athletes, and the public at-large. Sports related concussion (SRC) is generally considered to be a subset of mild traumatic brain injury (mTBI). mTBI remains a complicated clinical diagnosis due to highly variable symptom presentation involving multiple neurologic systems. A variety of neurological dysfunctions can be present (i.e. ocular, vestibular, cognitive, sleep, hormone imbalances, etc). The Center for Disease Control and Prevention estimates around 3.8 million TBIs occur in the USA annually. SRC has been discussed regularly in the popular press with celebrity athletes being allegedly impacted from a history of SRC, as well as lawsuits alleging brain injury from SRC. The health concerns and litigation has led to an increased concern regarding liability for athletic organizations and the implementation of concussion legislation across all 50 of the United States. Existing concussion legislation generally involves the following areas: (1) concussion education for nonmedical personnel such as coaches, parents/guardians, and athletes; (2) removal from play at the time of suspected injury without eligibility for same-day return-to-play; and (3) evaluation by a healthcare provider trained in TBI to diagnose an mTBI, or to clear an athlete to be eligible to continue playing.

The problem for performing step 3 above (evaluation by a healthcare provider to diagnose or clear the athlete; hereafter called “step 3”) is that there is no clear, science based, objective or quantitative method to perform that step. Those most experienced in performing such exams are neurologists and typical neurologists take weeks to get an appointment or need a referral from a clinician with a chief complaint or neurologic deficiency already identified, Currently, the people doing these evaluations are often athletic trainers, school nurses, neuro psychologists, chiropractors, or clinicians with similar skill sets. This evaluation and the skill of the evaluator is set forth in many of the states' legislations. The concern, for example, of an athletic trainer with a neuro course to make him or her eligible for doing a step 3 evaluation, is that they may not have the neurodiagnostic proficiency of a neurologist. What they need is a test panel, recognized by neurologists, to detect a concussion or document lack of suspicion of a concussion to allow return to play. There are no test panels published or marketed designed to aid in the step 3 evaluation.

Establishing neurologic wellness in a person without neurologic complaints is a difficult task for any clinician. Most clinicians are trained to identify an injury or disease, not to document an absence of injury or disease. There are multiple baseline and sideline tests and neurologists have years of experience to identify regions of the brain that are damaged post sports concussion. Baseline tests are unreliable because when people take them they often do not try their hardest, called “gaming the test,” making the tests utility questionable for step 3 evaluations. The computer-based baseline tests are also dependent upon the computer, computer screen, keyboard and mouse and changing these can change the results. This calls into question their utility for a step 3 tool. Further, there are protocols for return to play once a concussion has been diagnosed. These are distinct from an absence of suspicion when a concussion has not been diagnosed. A return to play post diagnosed concussion generally follows the symptoms of the patient. If no symptoms are reported there is lack of protocol for documenting a lack of suspicion in the presence of a lack of symptoms. It is common for those clinicians doing step 3 evaluations to have people present to them with no symptoms or concussion like complaints.

There is nothing in the current state of the art available to aid those clinicians in documenting an absence of suspicion and none of these tests or test panels are feasible for a step 3 evaluation.

### SUMMARY OF THE INVENTION

The absence of suspicion test of the present invention contains a test panel that addresses neurologic parameters and produces objective results needed to perform the step 3 evaluation. The invention can be performed by clinicians at the skill level usually present at initial evaluations and it is also sensitive enough to identify deficiencies to aid in the diagnosis of a concussion and or to raise suspicion such that the injured person could be referred to a higher level of medical care. The invention also produces results that if normal can be used to document an absence of suspicion of a concussion or traumatic brain injury and allow for safe return to play or regular activity. The current invention fills this need because the clinician can do a panel of neurologic tests that produce quantitative data and can make the proper diagnosis as to whether the patient has an injury that needs to be seen by a higher level Neurologist or is injury free and can safely return to normal activity.

In one embodiment, the present invention is a method of determining that an absence of suspicion of neurological injury is indicated in a subject. The method involves prompting the subject to engage in three or more of the following neurological assessments:
a. Reading Saccades,
b. Near/far accommodation using Hart charts of different font sizes
c. Brock string,
d. Maddox rod,
e. closed-eye turns,
f. Near Point of Convergence, and
g. PERRLA with consensual reflex.
   The data from the neurological assessments is used to determine that an absence of suspicion of neurological injury is indicated in the subject. In one embodiment, an absence of suspicion of neurological injury is indicated in the subject if, for each neurological assessment conducted, the test results meet the following criteria:
   Reading Saccades results are less than about 73 seconds;
   Near/far accommodation is greater than about 23 loops per minute;
   Brock string results are less than about 2 inches on either side of the target;
   Maddox rod vertical eye alignment is about 0 inches and horizontal eye alignment is less than about 2 inches to the left or right of the light;
   Closed-eye turns for the right turn and the left turn are from about 340 to about 380 degrees; Near Point of Convergence is less than about 3 inches; and
   PERRLA with consensual reflex is a normal reflex.

The test of the present invention can thereby document an absence of suspicion, allowing for safe return to play. Alternatively, a failure to “pass” one or more of the neurological assessments indicates a need for a higher level of assessment/care for the subject.

In another embodiment, the subject engages in four or more of the neurological assessments. In one embodiment, the subject engages in five or more of the neurological assessments. In another embodiment, the subject engages in six or more of the neurological assessments. In one embodiment, the subject engages in all of the neurological assessments.

In another embodiment, the method further includes the step of recording the results of the individual assessments on a document. In one embodiment, the method further includes the step of recording the results of the individual assessments in a computer-based data collection system. In another embodiment, the subject engages in the near/far accommodation test as one of the neurological assessments.

In one embodiment, the criteria for the Reading Saccades results are in the range of about 39 to about 72 seconds. In another embodiment, the criteria for the Near/far accommodation is from about 23 to about 37 loops called per minute. In one embodiment, the criteria for the Brock string results are from 0 to about 1.6 inches. In another embodiment, the criteria for the Maddox rod vertical eye alignment is about 0 inches and horizontal eye alignment is from about 0 to about 1.5 inches. In one embodiment, the criteria for the Closed-eye turns for a right turn are from about 348 to about 372 degrees and from about 345 to about 371 degrees for a left turn.

### BRIEF DESCRIPTION OF THE DRAWINGS

FIG. 1 is an embodiment of a patient consent form and data collection tool for the absence of suspicion test (AOST) of the present invention.

### DETAILED DESCRIPTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

Certain terminology is used in the following description for convenience only and is not limiting. As used herein, “absence of suspicion of neurological injury” means that there is negligible clinical evidence that a diagnosis of injury can be made.

Similar reference numerals will be utilized throughout the application to describe similar or the same components of each of the preferred embodiments of the implant described herein and the descriptions will focus on the specific features of the individual embodiments that distinguish the particular embodiment from the others

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As sports-related concussions gain notoriety, legislation has also increased, placing a greater liability on athletic trainers, who are typically the first to assess mild traumatic brain injury (mTBI). This has led to an increase in assessments for documenting an absence of or evidence of an mTBI. Although, their validation and standardization have been called into scrutiny. The present invention assists clinicians who are assessing an athlete following a suspected sports-related concussion (SRC). For example, the day after an athlete has a pull from play event, a clinician needs an objective system that will help direct care if needed and document an absence of suspicion of concussive signs. Such an objective system should be neurologic based, easy to perform, objective and quantitative.

The present invention discloses methods and data concerning the use of an absence of suspicion test (AOST) that can be utilized by athletic trainers and similarly trained clinicians to do an assessment after an athlete has presented with a suspected SRC. The test panel can be used to direct next steps concerning neurologic management as well as document brain health and allowing resumption of sport participation. In one embodiment, the AOST of the present invention is used to document absence of suspicion the day after an athlete has been pulled from play with a suspected mTBI, as is required by many state and local regulations.

### The AOST Panel

The individual assessments of the AOST tool of the present invention included: (1) Hart chart for saccadic eye movement, (2) Near/far accommodation using Hart charts of different font sizes, (3) Brock string for accommodation—convergence and suppression, (4) Maddox rod for horizontal and vertical phorias, (5) Closed-eye turns, for balance using a modified Rhomberg test, (6) Near point of convergence, and (7) Pupil response.

These methods for the individual assessments are described as follows:

Reading Saccades

Near/far accommodation using Hart charts of different font sizes

Brock String

Maddox Rod

Closed Eye Turns

Near Point of Convergence (NPC)

PERRLA with consensual reflex

### Reading Saccades

Method: The reading saccade test is done with a 10×10 alpha numeric called a Hart chart. Have the person read the sheet without moving their head. Moving only their eyes and not keeping track with their fingers. The chart should be at between 14 and 18 inches away and can be held or on a table. Tell the person what you are going to do and what they are going to be doing. The person calls out loud the alpha numeric until done. The order for calling them out is the top left character and then the top right character. Next the top left character, down one, and the top right character down one. The person alternates from the left column and right column going down until they reach the bottom and they move in one column. This continues until complete. It is best to demonstrate this by doing the first couple characters for them. You can switch to a spare sheet to avoid them memorizing the characters. Watch them during the test. They should not move their head, they must do every column and character. Be watchful for skipping columns. Time them and record how long it takes to complete the task. Results are reported in seconds. A “passing” assessment is indicated if the results are less than about 73 seconds. In another embodiment, a “passing” assessment is indicated if the results are in the range of about 39 to about 72 seconds

### Near/Far Accommodation Using Hart Charts of Different Font Sizes

Method: Have one sheet, the large sheet, on the wall at eye level. Stand 10 feet from the wall. Have the subject hold one sheet in their hand (smaller font) and read back and forth as described. Start top left near and go to top left far. Go down one and continue down the column. When the bottom of the column is reached move right one column. Time for 1 minute and record the score as a number of loops back and forth. Set up the large far alphanumeric card at eye level. Demonstrate where and how to hold the near card, just below the far card. Make sure that the far card is easily read at a distance of 10 feet. Ensure that the athlete does not need to squint to see the far card. Far card should be parallel to the ground from the person's eyes. The Near card slightly down so as to not obscure the far chart. Results are reported in a number of loops per minute. A “passing” assessment is indicated if the result is greater than about 23 loops per minute. In another embodiment, a “passing” assessment is indicated if the results are in the range from about 23 to about 37 loops called per minute.

### Brock String

Method: The brock string is a bead on a string. For this test it only needs one bead. Tell the person what you are going to do. Have them hold one end of the string to the bridge of their nose. Make sure their hand or fingers are not blocking either eye. Ensure their head is upright and gently pull the string straight away from their eyes and parallel to the ground. Your hand holding the string should be 14 inches away from the bead. Place the bead at about 14 inches from their nose. Say the following. “look at the bead. Is the bead in focus, does it look normal?” assuming the answers are yes, go on. Ask “when looking at the bead are you aware of two strings forming a shape like an Y, V, or X? If the answer is yes, ask “with the index finger not holding the string, point to where the strings cross, and keep your eyes on the bead.” Note in inches how far in front of the bead or past the bead they are pointing. If they point at the bead the score is 0. Results are reported in inches before or after the bead. A “passing” assessment is indicated if the result is less than about 2 inches on either side of the target. In another embodiment, a “passing” assessment is indicated if the results are in the range from 0 to about 1.6 inches.

### Maddox Rod

Method: The Maddox rod is a light splitter. It makes a light like a pen light or the light on a phone become a line and distorts the rest of the visual image. To use the Maddox rod you need a Maddox rod and a light source like a pen light. Place the penlight 20 feet away and at eye level. Tell the person what you are going to do. There is an orientation to the Maddox rod, make sure the ribs are parallel to the ground. Have the person look at the light and then place the Maddox rod in front of the right eye, keeping both eyes open. The subject should be holding the Maddox rod. Ask the following questions; “do you see the light? Do you see a vertical line? Is the line to the left, right or through the light?” If touching or though the light, record as 0. If they are separated, determine the number of inches left or right for the line. Using your finger or a pen ask them where the line is. You need to do this right next to the light source. Record number of inches RIGHT or LEFT for the right eye. The test can be repeated on the left eye. Make sure you document inches left and right AND the eye that has the Maddox rod. Rotate the Maddox rod such that the ribs are vertical up and down. Now the line will be horizontal. Repeat the steps above asking if the line is above or below the light. Record 0 for through or touching or number of inches above or below the light. Again, record which eye has the Maddox rod. This can be repeated on the other eye if needed. Results are reported in inches. A “passing” assessment is indicated if the result is about 0 inches for the vertical eye alignment and horizontal eye alignment is less than about 2 inches to the left or right of the light. In another embodiment, a “passing” assessment is indicated if the results are about 0 inches for the vertical eye alignment and from about 0 to about 1.5 inches for the horizontal eye alignment.

### Closed Eye Turns

Method: Have the person standing facing you. Tell them what you are going to do. Remind them to keep their eyes closed until you say to open them. State the following for part of the instructions. “Bring your feet together, stand up straight, head straight. Cross your arms across your chest and keep them there (demonstrate this). Close your eyes. Keep them closed.” Observe wobble or lean. “Now turn 360 degrees to your right.” Observe the turn. Document wobble. Report number of degrees turned. Tell them to open their eyes and relax for a moment. Make sure they are facing you again and repeat above for a left turn. Report number of degrees turned. A “passing” assessment is indicated if the results for the right turn and the left turn are from about 340 to about 380 degrees. In another embodiment, a “passing” assessment is indicated if the results are results for the right turn are from about 348 to about 372 degrees and from about 345 to about 371 degrees for the left turn.

### Near Point of Convergence (NPC)

Method: To determine the NPC have the person look at your finger or the tip of a pen. Their head should be straight up, not tilted or looking down. Your finger or tip of the pen should be about 2 feet away from them such that their eyes are straight and parallel to the floor. Tell them you are going to bring the finger or pen to the bridge of their nose and they are to track it and tell you if it changes: gets blurry, sees double, becomes uncomfortable. Slowly bring your finger or tip of the pen towards the bridge of the person's nose. Do not poke them or touch them. Watch their eyes. Note in inches any of the following: looking away, any complaints, one eye stops coming in, excessive blinking, leaning back. Record the number of inches from the bridge of the nose when something happens. Results are reported in inches. A “passing” assessment is indicated if the result is less than about 3 inches.

PERRLA with Consensual Reflex

Method: Perrla stands for pupils, equal, round, reacting to light and accommodating. The consensual reflex means the eye without the light constricts too. To perform and document this test make sure you are in an environment without bright direct sun light as this impairs the test results. Stand directly in front of the test subject and tell them what you are going to do. Use your phone or an eye light to shine the light in each eye TWICE. Keep the light on the eyes for at least 2 seconds. For this example, shine the eye light in the right eye and watch the right eye's pupil. Document: shape, and constriction to the light. A few moments later shine the light in the right eye again, but watch the left eye. The left eye should constrict as well. This is the consensual reflex. Repeat with the opposite eye. If both eyes constrict with light (on them and on the other eye), both eyes are round and both eyes have similar pupil sizes for each test the result is recorded as normal PERRLA with consensual reflex. A “passing” assessment is indicated if the result is a normal reflex.

In another embodiment, the subject engages in four or more of the neurological assessments. In one embodiment, the subject engages in five or more of the neurological assessments. In another embodiment, the subject engages in six or more of the neurological assessments. In one embodiment, the subject engages in all of the neurological assessments.

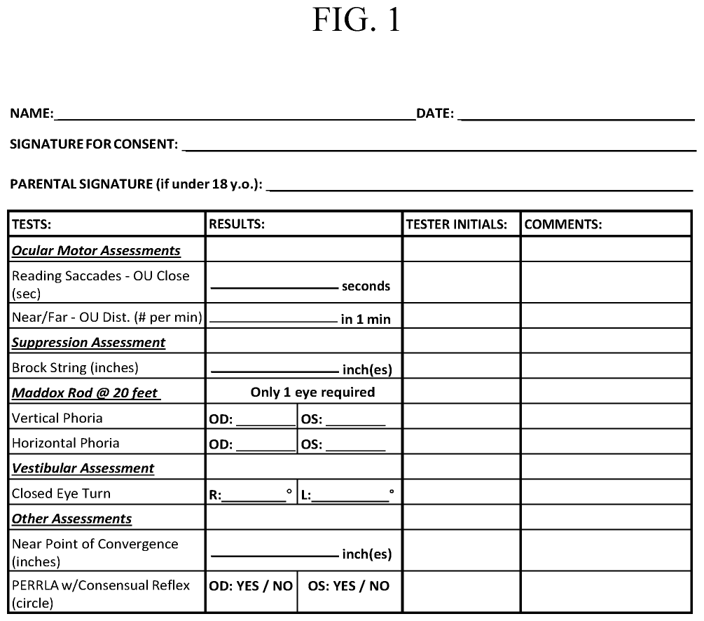

In one embodiment, the results of the individual assessments are recorded on a document such as the AOST data collection tool shown in FIG. 1. In another embodiment, the results of the individual assessments are recorded in a computer-based data collection system, such as an electronic database or a smartphone app.

The ‘absence of suspicion’ exam of the present invention is the first to demonstrate a series of non-computerized assessments designed to evaluate neurological functions that are commonly observed in individuals who are suspected to have incurred an SRC. One of the uses of this AOST tool is that it can be performed by trained athletic trainers as part of compliant SRC management and the test panel can be used to guide clinical management. In the current litigious environment, it is critical to document objective testing to safely return an athlete to play. The AOST results are objective and quantitative neurologic methods useful to make clinical decisions such as clear to play or aid in directing clinical SRC management.

The results presented below demonstrated that 72% of the AOST tested athletes had no evidence of an SRC. It is reported throughout the scientific literature that SRC symptoms may not present for several days. In these cases, the AOST was typically performed 24 to 48 hours post-incident. Of the 73.1% (n=19) determined to have an absence of suspicion, all returned to normal sporting activities and none reported the development of delayed post-concussive like symptoms. Moreover, 26.9% of athletes (n=7) tested were found to have a suspicious test during the AOST and were referred for neurologic exams. This was documented and those athletes were referred to UC's Independent Neurology Consultant for additional neurological evaluation. All 26.9% of those athletes (n=7) were eventually diagnosed with an SRC determined through a full neurological exam and then entered into the SRC management pathway.

The clinical observations noted during the AOST are also an important factor in making an SRC referral to a neuro specialist. In Table 2 we illustrate that the notes section of the AOST had good concordance (85.7%) with SRC referral and diagnosis of a concussion. These clinical notes are consistent with good clinical practice to take and record notes concerning diagnostic testing and management of the injured athlete. For example, with the Closed-Eye Turns balance assessment, five cases were noted where the turn was apparently normal in degrees turned, but deficiencies were noted including wobble, complaints of ‘spinning’ and or continued perception of rotation (dizziness). The Near/Far assessment was the most sensitive of the testing battery, with 57.1% of athletes to have incurred a suspicious injury (n=7) showing difficulty accommodating and elicited concerning symptoms common with concussion diagnoses.

The AOST of the present invention is designed to document and guide SRC related management shortly after a suspected concussive event; to document an absence of mTBI or recommend a full neurological assessment be performed. The AOST has utility for athletic trainers who want and/or need objective clinical documentation to return a player to play. Many of the states in the USA require documentation of an absence of SRC post pull from play event. The AOST can fulfill that test matrix and clinical documentation requirement concerning clearing an athlete and be compliant with most SRC protocols. It is important to realize that the AOST is distinct from a sideline or return to baseline assessment. The test results aid in communication with team physicians and SRC specialists. As the AOST battery is small in physical size and can easily fit within any regular travel bag or backpack, it may also be a useful tool for athletic trainers traveling with teams as well as managing SRCs. Thus, an athletic trainer traveling with a team without access to a neuro specialist or a qualified team physician, they can use the AOST to care for their athlete post suspected SRC. The AOST can be used in conjunction with other tests such as ImPACT, BESS and or SCAT. An experienced clinician can make their own decisions as to what methods to use when managing SRC and use the AOST to aid in documenting the SRC pathway.

The AOST tool is designed to be quick, quantitative and easy to perform. The cadre of tests are somewhat visually oriented but still multi-faceted in the functions tested. Including complimentary tests for a full AOST can be used by athletic trainers based on tests that they feel comfortable with. Tests that could be partnered with the AOST panel include but is not limited to; ImPACT, BESS, SCAT, NIH Memory Exam, Mini Mental Status Exam, Montreal Mental Status Exam, NIH Stroke Scale, CNS vital signs and others.

The AOST methods are a battery of neurological assessments which may be used by Athletic trainers to evaluate and document an absence of suspicion of injury. The data collected from using the AOST will help providers when making decisions to refer an athlete for further neurological testing. In addition, the AOST derives useful data to help guide clinical sports-related concussion management decisions.

In one embodiment, the method of the present invention can be used to document the absence of a mild traumatic brain injury or concussion. In another embodiment, the method of the present invention can be used to document brain normalcy when making a return to play decision. In one embodiment, the method of the present invention can be used to document brain normalcy when making a return to work or activity decision. Those experienced in the art will be able to substitute work for activities that one could be returning to where the AoST may aid in said decision making.

In another embodiment, the method of the present invention can be used to quantify brain health or normalcy following a suspected brain injury or concussion. In one embodiment, the method of the present invention can be used to quantify brain function following brain injury or stroke. Those experienced in the art will be able to suggest alternates concerning brain injury or stroke where the AoST may aid in said decision making. In another embodiment, the method of the present invention is a quantifiable method to be used by health care professionals when communicating about the health or normalcy of brain function.

In one embodiment, the method of the present invention is a quantifiable method that can be used by researchers to assess and or quantify brain health. In another embodiment, the method of the present invention is a quantifiable method that can be used by health care professionals managing a person with a suspected deficiency in brain health or normalcy. In one embodiment, the method of the present invention is a gateway method to aid health care professionals in guiding or recommending higher levels of care of a person or patient with a suspected deficiency in brain health or normalcy.

### Examples

A study was conducted involving 11 female and 15 male division-1 collegiate athletes between the ages of 18 and 22. The athletes were a part of a variety of sports teams including football, women's and men's soccer, women's volleyball, women's basketball, marching band, cheerleading, and dance. Information was collected on the AOST for 26 consecutive athletes who were referred to the colleges SRC team. All 26 athletes were referred by athletic trainers for evaluation of suspected SRC. This retrospective cohort study was reviewed and approved by the University of Cincinnati Institutional Review Board #2013-1534. The care or management was not changed for patients examined in this study. All personal and/or medical information has been removed.

### Results

The results were evaluated through descriptive statistics and reported as means, standard deviations and confidence intervals. Confidence intervals of 95% were chosen as a means by which practitioners could assess if their athletes were within a normal population.

Of the total 26 athletes, 19 athletes (73.1%) were found to have an absence of suspicion concerning SRC and were cleared to return to their sport. Seven athletes (26.9%) were found to have a suspicious exam resulting in a full neuro assessment. All 26.9% were eventually diagnosed with SRC. None of the 19 athletes who were cleared using the AOST tool were reported to have developed or experienced prolonged concussion-like symptoms.

Table 1 reports the averages, standard deviations, and confidence intervals (95%) of the individual assessments which make up the AOST. Within the AOST, the Near/Far test elicited concerning symptoms 57.1% of the time to indicate a suspected mTBI, having the greatest sensitivity of the individual tests. Also, clinical observations along with the AOST results accounted for or participated in 85.7% of the observations of an SRC referral for a full neuro exam (see Table 2). Table 2 lists the common clinical notes that were observed when there was a suspicion of an mTBI and referral for neuro exam.

TABLE 1
AOST Individual Test Descriptive Statistics

| | Average | STD | CI, 95% (±) |
|---|---|---|---|
| NPC^{a} (inches). | 1.26 | 1.66 | 0.789 |
| PERRLA w/consensual reflex^{b} | 1.00 | 1.00 | — |
| Reading saccades (seconds) | 55.6 | 16.7 | 7.51 |
| Near/far (# called per minute) | 30.3 | 6.80 | 3.70 |
| Brock string (inches) | −0.660 | 2.26 | 0.886 |
| Closed-eye turns-right (°) | 360 | 11.7 | 4.60 |
| Closed-eye turns-left (°) | 358 | 13.0 | 5.09 |
| Maddox rod-vert. eye | 0.00 | 0.00 | — |
| alignment (inches)^{c} | | | |
| Maddox rod-hor. eye | −0.103 | 1.65 | 0.783 |
| alignment (inches)^{c} | | | |
| AOST outcome: suspicion^{d} | 0.269 | 0.452 | 0.174 |

Table 1 shows descriptive statistics of AOST data collected from 26 collegiate athletes. ^{a}Near point of convergence. ^{b}1=normalreflex, 0=abnormal reflex. ^{c}Maddox rod values are measured from the inches off normal or “ortho” eye alignment position (parallel to ground), 0=ortho positioning, +=esophoric alignment, −=exophoric alignment. ^{d}Statistics of AOST exams resulting in a suspicion of injury determined and indicating referral to undergo full neurological assessment; 1=suspicion, 0=absence of suspicion.

TABLE 2

| Percentage with | |
|---|---|
| complaints | Common clinical notes |
| 57.1% | Difficulty accommodating during |
| | Near/Far assessment |
| 42.9% | Uncomfortable with NPC evaluation |
| 42.9% | Photophobic symptoms with PERRLA |
| 28.6% | Symptomatic with closed-eye turns |
| 28.6% | Neck pain and stiffness |

Table 2 shows the percentage of common clinical notes found when symptoms were 284 elicited from AOST examination on the 7 athletes determined to have a suspicion of concussive injury.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method of determining that an absence of suspicion of neurological injury is indicated in a subject, the method comprising prompting the subject to engage in three or more of the following neurological assessments:
a. Reading Saccades,
b. Near/far accommodation using Hart charts of different font sizes
c. Brock string,
d. Maddox rod,
e. Closed-eye turns,
f. Near Point of Convergence, and
g. PERRLA with consensual reflex; and
determining that an absence of suspicion of neurological injury is indicated in the subject if, for each neurological assessment conducted, the test results meet the following criteria:
Reading Saccades results are less than about 73 seconds;
Near/far accommodation is greater than about 23 loops per minute;
Brock string results are less than about 2 inches;
Maddox rod vertical eye alignment is about 0 inches and horizontal eye alignment is less than about 2 inches;
Closed-eye turns for the right turn and the left turn are from about 340 to about 380 degrees;
Near Point of Convergence is less than about 3 inches; and
PERRLA with consensual reflex is a normal reflex.

2. The method of claim 1 wherein the subject engages in four or more of the neurological assessments.

3. The method of claim 1 wherein the subject engages in five or more of the neurological assessments.

4. The method of claim 1 wherein the subject engages in six or more of the neurological assessments.

5. The method of claim 1 wherein the subject engages in all of the neurological assessments.

6. The method of claim 1 further comprising the step of recording the results of the individual assessments on a document.

7. The method of claim 1 further comprising the step of recording the results of the individual assessments in a computer-based data collection system.

8. The method of claim 1 wherein the subject engages in the Near/far accommodation neurological assessment.

9. The method of claim 1 wherein the subject engages in the Closed-eye turns neurological assessment.

10. The method of claim 1 wherein the criteria for the Reading Saccades results are in the range of about 39 to about 72 seconds.

11. The method of claim 1 wherein the criteria for the Near/far accommodation is from about 23 to about 37 loops called per minute.

12. The method of claim 1 wherein the criteria for the Brock string results are from 0 to about 1.6 inches.

13. The method of claim 1 wherein the criteria for the Maddox rod vertical eye alignment is about 0 inches and horizontal eye alignment is from about 0 to about 1.5 inches.

14. The method of claim 9 wherein the criteria for the Closed-eye turns for a right turn are from about 348 to about 372 degrees and from about 345 to about 371 degrees for a left turn.
